(19) United States
(12) Patent Application Publication
(10) Pub. No.: US 2011/0300073 A1
(43) Pub. Date: Dec. 8, 2011

(54) 18F-LABELLED THREE-AND FOUR-CARBON ACIDS FOR PET IMAGING

(75) Inventors: Nagavarakishore Pillarsetty, Jackson Heights, NY (US); Steven Larson, New York, NY (US)

(73) Assignee: SLOAN-KETTERING INSTITUTE FOR CANCER RESEARCH, New York, NY (US)

(21) Appl. No.: 12/999,120

(22) PCT Filed: Jun. 15, 2009
(86) PCT No.: PCT/US09/47338

§ 371 Date: Apr. 1, 2011

Related U.S. Application Data
(60) Provisional application No. 61/132,328, filed on Jun. 16, 2008.

(51) Int. Cl. A61K 51/04; C12N 5/09

(52) U.S. Cl. 424/1.89; 435/375

(57) ABSTRACT

Compositions containing three and four-carbon acids labeled with ¹⁸F at the 2-position and to their use for emission tomography are disclosed.

### CROSS REFERENCE TO RELATED APPLICATIONS

This application claims priority from U.S. provisional application 61/132,328, filed Jun. 16, 2008, the entire disclosure of which is incorporated herein by reference.

### FIELD OF THE INVENTION

The invention relates to compositions containing three and four-carbon acids labeled with ¹⁸F at the 2-position and to their use for emission tomography.

### BACKGROUND OF THE INVENTION

Medical radionuclide imaging is a key component of modern medical practice.

This methodology involves the administration, typically by injection, of tracer amounts of a radioactive substance, which subsequently localize in the body in a manner dependent on the physiologic function of the organ or tissue system being studied. The radiotracer emissions, most commonly gamma photons, are imaged with a detector outside the body, creating a map of the radiotracer distribution within the body. These images provide information of great value in the clinical diagnosis and treatment of disease.

Recent advances in diagnostic imaging, such as magnetic resonance imaging (MRI), computerized tomography (CT), single photon emission computerized tomography (SPECT), and positron emission tomography (PET) have made a significant impact in cardiology, neurology, oncology, and radiology. Although these diagnostic methods employ different techniques and yield different types of anatomic and functional information, this information is often complementary in the diagnostic process.

The field of non-invasive molecular imaging using PET isotopes has made rapid advances for the detection of primary and metastatic tumors. The agents for PET imaging are commonly labeled with positron-emitters, such as ¹¹C, ¹³N, ¹⁵O, ¹⁸F, ⁷⁵Br, ⁷⁶Br and ¹²⁴I. [¹⁸F]-Fluorodeoxyglucose ([¹⁸F]-FDG) has been shown to be an effective tumor imaging agent. However, its development has been very slow in the area of prostate cancer. Prostate cancer is the most commonly diagnosed form of cancer in men and second leading cause of cancer deaths in men in United States. Early detection of prostate tumors is of extreme importance for successful outcome of the treatment, because there are no successful treatment options for metastatic prostate tumors. Even for non-metastatic prostate tumors, the precise location and target definition is critical for defining clinical target volume for conformal radiotherapy. Hence, there is an urgent need to develop PET imaging agents that can be routinely used for detection and characterization of prostate tumors.

[¹¹C]-Acetate initially was developed as a PET tracer to measure myocardial metabolism, but it was also found to be effective in detecting prostate tumors. Though the mechanism of uptake has been unclear, it was recently shown that most prostate tumors over-express Fatty Acid Synthase, which uses acetate as its substrate for the synthesis of long chain fatty acids, and it has been hypothesized that this is the reason that increased uptake of [¹¹C]-acetate is observed in tumors. However, although [¹¹C]-acetate does allow one to visualize prostate tumors, the short half-life of carbon-11 (20 min.) usually requires a cyclotron on site, which is something of an impediment to its use as a routine PET imaging agent. On the other hand, because of their relatively long half-life (2 hours), fluorine-18 labeled tracers do not require an onsite cyclotron. For that reason, [¹⁸F]-fluoroacetate and [¹⁸F]-fluorine derivatives of choline are currently being investigated as prostate tumor imaging agents. [¹⁸F]-Fluoroacetate mimics [¹¹C]-acetate in the primary steps and has been shown to accumulate in prostate tumors, but unfortunately it also exhibits high toxicity. Choline derivatives might be considered as PET diagnostic agents for prostate cancer, but low sensitivity—is a drawback to the wide applicability of [¹⁸F]-fluorine derivatives of choline. Hence, there is an unmet need to develop new PET tracers which can overcome the drawbacks or complement current tracers. PET imaging agents, particularly to image the prostate, an organ for which there are not, currently, good imaging compounds would be of great value for diagnostic and prognostic purposes.

2-[¹⁸F]-Fluoropropionic acid (2-[¹⁸F]-FPA) is known as a starting material for attaching a labeled fluorine-containing residue to peptides. In fact, 2-[¹⁸F]-FPA is used as a starting material in the chemical synthesis of ¹⁸F-Galacto-RGD, which is currently under clinical trials for imaging αᵥβ₃ integrin expression. However, the biodistribution and imaging properties of 2-[¹⁸F]-FPA itself have apparently never been reported in the literature.

### SUMMARY OF THE INVENTION

In one aspect, the invention relates to diagnostic compositions comprising a pharmaceutical carrier for injection and a 2-[¹⁸F]-fluoro C3 or C4 acid.

In another aspect, the invention relates to the use of a 2-[¹⁸F]-fluoro C3 or C4 acid for positron emission tomographic imaging.

In another aspect, the invention relates to methods for detecting abnormalities in a tissue or organ of a mammal. A method comprises: (1) administering to the mammal an amount of a 2-[¹⁸F]-C3 or C4 acid sufficient to be detected by a nuclear imaging technique (2) forming at least one image showing the distribution of the 2-[¹⁸F]-C3 or C4 acid within the tissue or organ of the mammal by nuclear imaging; and (3) detecting the abnormality by observing the image.

In another aspect, the invention relates to methods for rendering neoplastic tissue visible by positron emission tomography (PET). A method comprises delivering to the tissue an amount of a 2-[¹⁸F]-C3 or C4 acid sufficient to be detected by positron emission tomography.

### BRIEF DESCRIPTION OF THE FIGURES

FIG. 1 is a pair of MicroPET images of nude mice with CWR22RV1 xenografts imaged with either ²-[¹⁸F]-FPA or [¹⁸F]-FDG one hour after tail vein injection. The images, which would normally be presented in color because of its higher information value, have been rendered in black and white to meet the requirements of PCT Rule 11.13.

### DETAILED DESCRIPTION OF THE INVENTION

It has now been found that 2-[¹⁸F]-fluoropropanoic acid (which could also be called 2-[¹⁸F]-fluoropropionic acid), 2-[¹⁸F]-fluorobutanoic acid (which could also be called 2-[¹⁸F]-fluorobutyric acid) and 2-[¹⁸F]-fluoro-2-methylpropionic acid (which could also be called 2-fluoroisobutyric acid):

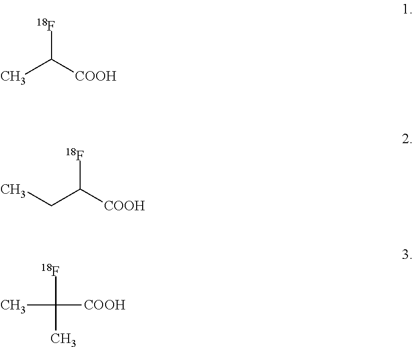

are versatile PET tracers for imaging xenografts of prostate tumors and other tumors in mice and, by extension, in humans and other mammals.

Dreisbach's Handbook of Poisoning, 13^{th} Edition (True and Dreisbach, Informa Health Care, 2002) sets forth an oral LD₅₀ in rats of 0.22 mg/kg for 2-fluoroacetic acid. In contrast, 2-fluoropropanoic acid was given at 212 mg/kg to rats and no toxicity was observed. The thousand-fold improvement in oral LD₅₀ on going from acetate to propionate might be due to the known interference of fluoroacetic acid in the Krebs cycle via a pathway that is inaccessible to propionic acid. Whatever the underlying mechanism, the discovery that 3 and 4-carbon acids provide very good PET images makes possible an enormous improvement in therapeutic index over the known 2-[¹⁸F]-fluoroacetic acid.

As will be apparent to the person of skill, 2-fluoropropanoic acid (1) and 2-fluorobutanoic acid (2) can exist as enantiomers. Unless otherwise stated or depicted, structures depicted herein are meant to include all stereoisomeric (e.g., enantiomeric) forms of the structure, for example, the R and S configurations for the asymmetric center as in 1a and 1b:

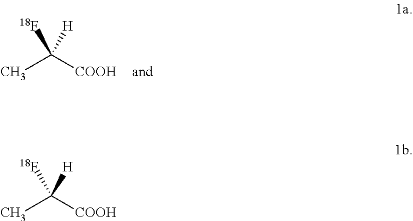

as well as a mixture of any such forms of that compound in any ratio. Therefore, single, pure enantiomers, racemates—and any ratio in between—are within the scope of the invention.

As used herein, and as would be understood by the person of skill in the art, the recitation of an acid—unless expressly further limited—is intended to include salts of that acid. Thus, for example, the recitation “2-[¹⁸F]-fluoropropanoic acid” would include salts

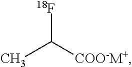

wherein M is any counterion, particularly a pharmaceutically acceptable counterion. The term “pharmaceutically acceptable salt” refers to salts prepared from pharmaceutically acceptable non-toxic bases including inorganic bases and organic bases. Suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations, which may be attached to alkyl having from 1 to 20 carbon atoms or may be NH₄⁺.

Compositions of the invention comprise a pharmaceutical carrier for injection and one or more of the 2-[¹⁸F]-fluoro C3 or C4 acids (or, as explained above, a pharmaceutically acceptable salt of the acid). The compositions may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The pharmaceutical carrier may be physiologic saline (0.9%) or phosphate buffered saline. The composition may additionally comprise a stabilizer. Chemical stabilizers are useful to reduce the likelihood for radiolysis-induced decomposition of the ¹⁸F-labeled compound at high radioactivity concentrations. Suitable stabilizers include antioxidants such as the pharmaceutically acceptable antioxidant, sodium L-ascorbate.

The compositions are useful for positron emission tomography of various organs and tissues including prostate, blood, lymph, ovary, cervix, bladder, breast, liver, kidney, heart and brain, particularly for positron emission tomography of prostate.

In a method aspect, the invention relates to a method for detecting abnormalities in a tissue or organ of a mammal. The method comprises (1) administering a 2-[¹⁸F]-C3 or C4 acid; (2) forming at least one image showing the distribution of the 2-[¹⁸F]-C3 or C4 acid within the tissue or organ of the mammal by nuclear imaging; and (3) detecting the abnormality by observing the image. The abnormality can be detected by comparing the image of the suspected abnormality with an image showing the normal concentrations and distribution of the 2-[¹⁸F]-fluoro C3 or C4 acid in the tissue or organ of mammals of the same species. This step is optional because in many, if not most, cases, normal tissue will be invisible or faintly visible in the PET scan whereas abnormal (neoplastic) tissue will be highly visible, and an actual comparison step is not necessary for each evaluation or detection. An effective amount of 2-[¹⁸F]-C3 or C4 acid is commonly from 100 μCi to 50 mCi. The nuclear imaging technique may be positron emission tomography (PET) or single photon emission computed tomography (SPECT). The abnormality will often be neoplastic tissue and the tissue will be found in a prostate, a breast or a brain, particularly a tumor in a prostate.

In another method aspect, the invention relates to method for rendering neoplastic tissue visible by positron emission tomography (PET). The method comprises delivering to the tissue an amount of a 2-[¹⁸F]-C3 or C4 acid sufficient to be detected by positron emission tomography. The acid may be delivered to the organ or tissue ex vivo by flushing with a composition as described above. The acid may be delivered in vivo by injecting a composition as described above into a blood vessel or tissue of a mammal. The acid may also be delivered in vivo by injecting a composition comprising a biological precursor of the acid into a blood vessel or tissue. For example, one could inject a methyl or ethyl ester (e.g. methyl 2-[¹⁸F]-fluoropropanoate or ethyl 2-[¹⁸F]-fluoropropanoate) into the mammal and allow the mammal's esterases to cleave the ester to the acid. Alternatively, one could inject an amide or N-methylamide (e.g. 2-[¹⁸F]-fluoropropanamide or N-methyl-2-[¹⁸F]-fluoropropanamide) into the mammal and allow the mammal's amidases to cleave the amide to the acid. Alternatively, one could inject a β-¹⁸-fluoro-α-keto-4- or 5-carbon acid into the mammal and allow the mammal's pyruvate dehydrogenase system to remove the elements of carbonyl to provide the 2-[¹⁸F]-C3 or C4 acid.

2-[¹⁸F]-Fluoropropionic Acid was synthesized as follows:

All reagents and solvents were purchased either from Aldrich Chemical Company (St. Louis, Mo.) or Fisher Scientific (Pittsburgh, Pa.) and were used without further purification, unless stated otherwise. All HPLC solvents were filtered (0.45 μm, nylon, Alltech) prior to use. Water (ultra-pure, ion-free) was obtained from a Millipore Alpha-Q Ultra-pure water system. HPLC was performed using a Shimadzu (Columbia, Md.) system composed of a C-18 reversed-phase column (Phenominex Luna analytical 4.6×250 mm or semi-prep 10×250 mm, 5 μL, 1.0 or 4.0 mL/min, 0.2% Acetic acid/CH3CN), two LC-10AT pumps, an SPD-M10AVP photodiode array detector and a BioScan Flow Count radiodetector using a 25×25 mm NaI(Tl) crystal. Radioactivity was assayed using a Capintec CRC-15R dose calibrator (Ramsey, N.J.).

No-carrier-added [¹⁸F] fluoride ion was produced by the ¹⁸O(p,n)¹⁸F nuclear reaction by bombardment of an enriched [¹⁸O]H₂O target with 11 MeV protons using an EBCO-TR19 cyclotron.

2-[¹⁸F]-Fluoropropionic Acid was synthesized using an established procedure (Scheme 1).

Briefly, ¹⁸F-Fluoride ion (2.8 GBq, 75 mCi) in water, was added to a vial containing 80 μl of 0.25M potassium carbonate and 13 mg of 4,7,13,16,21,24-hexaoxa-1,10-diazabicyclo[8.8.8]-hexacosane (sold under the tradename KRYPTOFIX®) dissolved in 1 ml acetonitrile. Water was removed azeotropically with acetonitrile (3×1 mL) at 80° C. To the anhydrous [¹⁸F]KF/K₂CO₃ complexed with Kryptofix was added a solution of methyl-2-d,l-bromopropionate (3 mg) in 300 μL of anhydrous acetonitrile and the vial was sealed and heated at 80° C. for 4 minutes to give methyl-2-[¹⁸F]-fluoropropionate. The vial was allowed to cool to room temperature and diluted with 700 μL of water and injected into HPLC for purification. Purification was carried out on semi prep Luna-C18 column using 60 (0.2% acetic acid)/40 acetonitrile solvent as eluant. The product, methyl-2-[¹⁸F]-fluoropropionate, fraction was collected and to it 50 μl of 10N NaOH was added and heated at 80° C. for 10 minutes to give sodium 2-[¹⁸F]-fluoropropionate. The solvent was removed under reduced pressure and the residue was neutralized with 85 μl of 6N HCl. The product was formulated in 0.9% saline and used for studies. For the analysis of purity of the product, the 40 μl of product was acidified with 10 μl of 1N HCl and analyzed using C-18 Luna column with 100 (0.2% acetic acid) as eluting solvent. The final yield was about 50% (unoptimized).

The overall synthesis time for 2-[¹⁸F]-FPA was 45 minutes and the average yields obtained were about 50% (decay corrected). The purity was at least >95%. Due to absence of chromophore, there was no absorption in the UV spectrum. Hence, the average specific activity was calculated based on total conversion of the precursor to the fluorinated derivative. The average specific activity estimated was greater than 0.7 GBq/μMol.

2-[¹⁸F]-Fluorobutanoic acid and 2-[¹⁸F]-fluoro-2-methylpropionic acid may be made in similar fashion starting from the appropriate α-bromoester. The overall synthesis time for 2-[¹⁸F]-FBA was 80 minutes and the average yields obtained were about 50% (decay corrected). If one wanted to employ the esters in vivo as described above, one would omit the saponification step. If one wanted to employ the amides in vivo as described above, one would convert the acids or esters by methods well-known in the art.

Small animal PET imaging was performed on MicroPET, to evaluate the potential of 2-[¹⁸F]-fluoropropanoic acid (2-[¹⁸F]-FPA), and 2-[¹⁸F]-fluorobutanoic acid (2-[¹⁸F]-FBA) as tumor imaging agents in mice with prostate cancer CWR22RV1 xenografts. One hour post administration the tumor is easily visualized in both transaxial and coronal slices with 2-[¹⁸F]-FPA in a MicroPET image with 5 minute acquisition time. In addition to uptake in the tumor, there is high uptake in heart and brain.

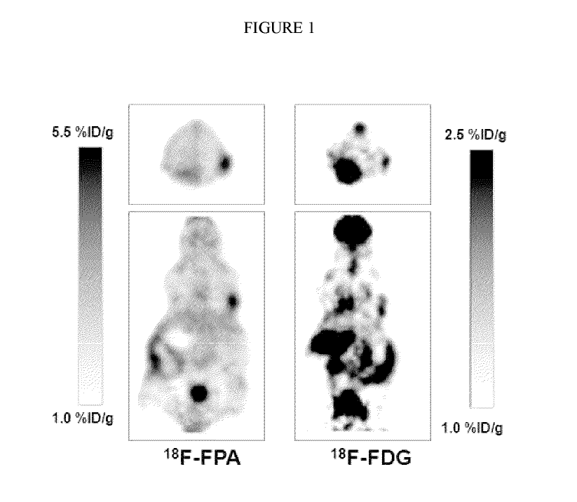

FIG. 1 shows MicroPET images of mice imaged with 2-[¹⁸F]-FPA and [¹⁸F]-fluorodeoxyglucose ([¹⁸F]-FDG) in order to compare one of the compounds of the present invention with other imaging compounds. The mice were imaged using MicroPET first with [¹⁸F]-FDG after 4 hour fasting 1 hour post injection. The activity was allowed to decay for 20 hrs and the same mice were imaged with 2-[¹⁸F]-FPA 1 hour post injection. In case of 2-[¹⁸F]-FPA, the animals were not fasted. As shown in FIG. 1, the tumors can be visualized much more clearly with 2-[¹⁸F]-FPA as compared to [¹⁸F]-FDG. 2-[¹⁸F]-FPA has high uptake in brain like [¹⁸F] FDG, but, unlike [¹⁸F]-FDG there is much lower accumulation in the kidneys of mice.

All animal experiments were done in accordance with protocols approved by the Institutional Animal Care and Use Committee of Memorial Sloan-Kettering Cancer Center and followed National Institutes of Health guidelines for animal welfare. Subcutaneous tumors were produced in nude mice (20-25 g; Charles River Laboratories) by subcutaneous injection of 5×10⁶ tumor cells in 200 μl consisting of 100 μl of cell culture medium and 100 μl of Matrigel under 2% Isofluorane anesthesia on the right forelimb of mice.

Imaging was performed by use of a dedicated small-animal PET scanner (Focus 120 microPET; Siemens Medical Solutions USA, Inc.). Mice were maintained under 2% isoflurane anesthesia in oxygen at 2 L/min during the entire scanning period. Imaging was performed one hour post administration of 11.1 MBq (300 μCi) of either 2-[¹⁸F]-FPA or [¹⁸F]-FDG via the lateral tail vein. An energy window of 350-700 keV and a coincidence timing window of 6 ns were used. The image data were corrected for non-uniformity of the scanner response, dead time count losses, and physical decay to the time of injection. No correction was applied for attenuation, scatter, or partial-volume averaging. The measured reconstructed spatial resolution of the Focus 120 scanner is ˜1.6 mm full width at half maximum at the center of the field of view.

For single isotope ([¹⁸F]) biodistribution studies, mice with CWR22rv1 tumors were injected intravenously in the tail vein with 3.7-5.5 MBq (100-150 μCi) of [¹⁸F]-FPA in 200 μL of saline. Radioactivity in the syringe before and after administration was measured in an energy-calibrated dose calibrator (CRC-15R; Capintec) and exact quantity received by each animal was determined. The animals were euthanized at different time points and then the organs were harvested. [¹⁸F] radioactivity was measured in a calibrated gamma counter (Perkin Elmer 1480 Wizard 3 Auto Gamma counter, Waltham, Mass.) using 400-600 keV energy window and decay correction. The counts were converted into activity and % ID/g was calculated by dividing with decay corrected injected activity and weight of the organ. The in vivo biodistribution profile of 2-[¹⁸F]-FPA at 1, 2, 3 and 4 hour post administration via tail vein in nude mice with CWR22RV1 xenografts was examined, and it was found that biodistribution remains similar during the time of study. There is considerable accumulation of tracer in tumor. In addition to tumor, there is high uptake in blood and heart. The tumor to organ ratio of 2-[¹⁸F]-FPA at 1, 2, 3 and 4 hour post injection is:

| organ | 1 hour | 2 hours | 3 hours | 4 hours |
|---|---|---|---|---|
| blood | 1.05 | 1.03 | 1.04 | 1.06 |
| tumor | 1.00 | 1.00 | 1.00 | 1.00 |
| heart | 0.90 | 0.94 | 1.07 | 1.00 |
| lungs | 1.22 | 1.17 | 1.30 | 1.32 |
| liver | 1.35 | 1.24 | 1.33 | 1.41 |
| spleen | 1.40 | 1.56 | 1.62 | 1.56 |
| stomach | 2.50 | 3.60 | 4.48 | 3.54 |
| small intestine | 1.26 | 1.57 | 1.58 | 1.44 |
| large intestine | 1.39 | 1.49 | 1.51 | 1.87 |
| kidneys | 1.43 | 1.57 | 1.60 | 1.29 |
| muscle | 1.79 | 1.79 | 1.76 | 1.85 |
| bone | 2.79 | 2.14 | 2.58 | 1.90 |
| spine | 1.76 | 1.61 | 1.57 | 1.44 |

The tumor to organ ratio is always greater than 1 for most of the organs except for heart which is around 0.95.

With other PET imaging compounds typically, the patient is fasted at least 4 hours prior to administration of the analog. An additional advantage of the imaging compound of the present invention is that the patient does not need to fast prior to administration.

The radiation dose estimates to human organs is determined from calculations based on the 2-[¹⁸F]-FPA biodistribution data in mice according to methods known to a person skilled in the art. In order to produce conservative estimates, the total body residence time is assumed to be determined only from radioactive decay (1.44.times.half-life=2.6 hr).

The imaging compounds of the present invention can be used in the detection and localization of a wide variety of neoplasms, including but not restricted to prostate cancer, breast cancer, and lymphomas. The analogs are particularly useful for imaging pelvic tumors (the pelvis being defined as that region that extends from the bottom of the ishia to the top of the iliac crest), including prostate tumors and metastases of prostate tumors in the pelvic lymph nodes, ovarian cancer, cervical cancer and bladder cancer. (Should we include a statement about blood flow)

The compounds of the present invention can also be used to guide the biopsy of malignancies and monitor the effects of various therapeutic regimens, including chemotherapy. In accordance with the present invention, neoplasms can be detected and localized in the context of oncologic surgical procedures using an intraoperative radioactivity detection probes. The patient can be administered the ¹⁸F-labeled analog and an appropriately shielded radiation detector can be subsequently used during the surgical procedure to detect and/or localize neoplasm(s) in the body, such as to identify lymph nodes that bear malignant tissue. When the method is performed in the pelvic region, there may be an advantage to urethral catheterization and irrigation of the urinary bladder in order to reduce the confounding radioactivity in urine.

The compounds of the present invention can also be used in the noninvasive assessment of the response of neoplastic tissue in a patient to therapeutic interventions using PET scanning or another external radiation detection technique. The patient can be scanned at more than one time and the data from two or more scans are compared to determine potential differences in the tumor uptake of the analog.

The compounds of the present invention can also be used in the staging of neoplasms based on quantitative or qualitative measurements of uptake of the present analogs by tissue. The tissue uptake of the analog can be determined while the tissue is within the body or outside the body. The uptake measurements can be performed in conjunction with pathologic, histologic, histochemical and/or immunohistochemical assessment of the same tissue for classification and evaluation of malignancy. The method of the present invention can be used to determine the degree of malignancy of a tissue by quantitating the amount of ¹⁸F radioactivity present.

The compounds of the present invention can also be used in the anatomical mapping of the distribution of neoplastic tissue in the body using PET or another external radiation detection technique in combination with anatomical images obtained using CT, MRI, or ultrasound. The anatomical images can be acquired using a dedicated CT/PET, MRI/PET, PET/ultrasound scanning device or separate PET and CT/MRI/ultrasound scanning devices. If separate PET and CT/MRI/ultrasound imaging devices are used, image analysis techniques can be employed to spatially register the PET images with the anatomical images. The method can be used for intraorgan mapping of neoplastic tissue, for example, the spatial distribution of prostate carcinoma within the prostate gland can be determined for aiding in biopsy of the prostate gland or planning of radiation therapy of the prostate gland either by external beam radiation or brachytherapy. Likewise, the method may be used for guiding the biopsy or surgical resection of lymph nodes.

1. A diagnostic composition comprising a pharmaceutical carrier for injection and a 2-[¹⁸F]-fluoro C3 or C4 acid.

2. A composition according to claim 1 wherein said pharmaceutical carrier for injection comprises physiologic saline or phosphate buffered saline.

3. A composition according to claim 2 additionally comprising a stabilizer.

4. (canceled)

5. A composition according to claim 1 wherein said C3 or C4 acid is propanoic acid.

6. A composition according to claim 1 wherein said C3 or C4 acid is butanoic acid.

7. A composition according to claim 1 wherein said C3 or C4 acid is 2-methylpropanoic acid.

8.-14. (canceled)

15. A method for detecting abnormalities in a tissue or organ of a mammal, said method comprising (1) administering to said mammal an amount of a 2-[¹⁸F]-C3 or C4 acid sufficient to be detected by a nuclear imaging technique (2) forming at least one image showing the distribution of the 2-[¹⁸F]-C3 or C4 acid within the tissue or organ of the mammal by nuclear imaging; and (3) detecting the abnormality by observing the image.

16. A method according to claim 15 wherein detecting the abnormality is carried out by comparing the image with an image showing the normal concentrations and distribution of the 2-[¹⁸F]-fluoro C3 or C4 acid in the tissue or organ of mammals of the same species.

17. A method according to claim 15, wherein the effective amount of 2-[¹⁸F]-C3 or C4 acid is from 100 μCi to 50 mCi.

18. A method according to claim 15, wherein the nuclear imaging technique is selected from positron emission tomography (PET) and single photon emission computed tomography (SPECT).

19. A method according to claim 15 wherein said abnormality is neoplastic tissue.

20. A method according to claim 15 for detecting a tumor in a prostate, a breast or a brain.

21. A method according to claim 20 for detecting a tumor in a prostate.

22. A method according to claim 15 wherein said C3-C4 acid is 2-[¹⁸F]-fluoropropanoic acid.

23. A method according to claim 15 wherein said C3-C4 acid is 2-[¹⁸F]-fluorobutanoic acid.

24. A method according to claim 15 wherein said C3-C4 acid is 2-[¹⁸F]-fluoro-2-methylpropanoic acid.

25. A method for rendering neoplastic tissue visible by positron emission tomography (PET) said method comprising delivering to said tissue an amount of a 2-[¹⁸F]-C3 or C4 acid sufficient to be detected by positron emission tomography.

26. An in vivo method according to claim 25 wherein said tissue is in a living mammal and delivering 2-[¹⁸F]-C3 or C4 acid to said tissue is accomplished by injecting a composition according to claim 1 into a blood vessel of said mammal.

27. An ex vivo method according to claim 25 wherein delivering 2-[¹⁸F]-C3 or C4 acid to said tissue is accomplished by flushing said tissue with a composition according to claim 1.

28. A method according to claim 25 wherein said tissue is prostate, blood, lymph, ovary, cervix, bladder, breast or brain tissue.

29. A method according to claim 28 wherein said tissue is prostate tissue.

30. A method according to claim 29 wherein said C3-C4 acid is 2-[¹⁸F]-fluoropropanoic acid.
